(19) United States
(12) Patent Application Publication
(10) Pub. No.: US 2021/0400440 A1
(43) Pub. Date: Dec. 23, 2021

(54) SYSTEM FOR TRACING REGISTERED AND NON-REGISTERED DEVICES

(71) Applicant: Intrex, Reston, VA (US)

(72) Inventor: Ted Tzirimis, Reston, VA (US)

(21) Appl. No.: 17/352,955

(22) Filed: Jun. 21, 2021

Related U.S. Application Data
(60) Provisional application No. 63/041,085, filed on Jun. 18, 2020.

(51) Int. Cl. H04W 4/029; H04B 17/318; H04B 17/27; G16H 50/80

(52) U.S. Cl. CPC H04W 4/029; G16H 50/80; H04B 17/27; H04B 17/318

(57) ABSTRACT

A computer-implemented method includes: receiving, by a computing device, a first type of communication signal data object comprising an identifier of a registered user device, an identifier of a network device, and a measure of signal strength of a communication signal emitted by the registered user device and received by the network device; determining, by the computing device, a location of the registered used device based on a known location of the network device and the measure of the signal strength; generating, by the computing device, a first location data record identifying the determined location of the registered used device and a user registered to the registered user device; and storing or outputting, by the computing device, the first location data record.

### CROSS-REFERENCE TO RELATED APPLICATIONS

This application claims priority to U.S. provisional patent application No. 63/041,085 filed on Jun. 18, 2020, which is hereby incorporated by reference in its entirety.

### BACKGROUND

A user device may include a computing device capable of communicating via a network. For example, a user device may include to a mobile communication device (e.g., a smart phone or a personal digital assistant (PDA)), a portable computer device (e.g., a laptop or a tablet computer), a wearable device (e.g., a smart watch, fitness band, smart eyewear, smart clothing, etc.).

A Global Position System (GPS) is a satellite-based radio-navigation that provides geolocation and time information to a GPS receiver anywhere on or near the Earth where there is an unobstructed line of sight to four or more GPS satellites. GPS location accuracy may be based on the strength of the GPS signal received by a receiver. Obstacles such as mountains and buildings block or diminish the strength of GPS signals, thus reducing location accuracy.

Internet of Things (IoT) is a system of interrelated computing devices (e.g., user devices, as described herein), mechanical and digital machines provided with unique identifiers (UIDs) and the ability to transfer data over a network without requiring human-to-human or human-to-computer interaction. Any variety of IoT devices may be connected to form a smart network in a facility, home, etc.

### SUMMARY

In one example aspect, a computer-implemented method includes: receiving, by a computing device, a first type of communication signal data object comprising an identifier of a registered user device, an identifier of a network device, and a measure of signal strength of a communication signal emitted by the registered user device and received by the network device; determining, by the computing device, a location of the registered used device based on a known location of the network device and the measure of the signal strength; generating, by the computing device, a first location data record identifying the determined location of the registered used device and a user registered to the registered user device; and storing or outputting, by the computing device, the first location data record.

In another example, a computer program product is disclosed that comprises a computer readable storage medium having program instructions embodied therewith, the program instructions executable by a computing device to cause the computing device to perform a method comprising: receiving, by the computing device, a first type of communication signal data object comprising an identifier of a registered user device, an identifier of a network device, and a measure of signal strength of a communication signal emitted by the registered user device and received by the network device; determining, by the computing device, a location of the registered used device based on a known location of the network device and the measure of the signal strength; generating, by the computing device, a first location data record identifying the determined location of the registered used device and a user registered to the registered user device; and storing or outputting, by the computing device, the first location data record.

In yet another example, a system is disclosed that comprises a processor, a computer readable memory, a non-transitory computer readable storage medium associated with a computing device, and program instructions executable by the computing device to cause the computing device to perform operations of a method comprising: receiving, by a computing device, a first type of communication signal data object comprising an identifier of a registered user device, an identifier of a network device, and a measure of signal strength of a communication signal emitted by the registered user device and received by the network device; determining, by the computing device, a location of the registered used device based on a known location of the network device and the measure of the signal strength; generating, by the computing device, a first location data record identifying the determined location of the registered used device and a user registered to the registered user device; and storing or outputting, by the computing device, the first location data record.

Various additional features can be included in the above-noted examples including one or more of the following features. The method further comprises receiving a second type of communication signal data object comprising an identifier of a non-registered user device, an identifier of the network device, and a measure of signal strength of a communication signal emitted by the non-registered user device and received by the network device; determining a location of the non-registered user device based on the known location of the network device and the measure of the signal strength of the communication signal emitted by the non-registered user device and received by the network device; generating a second location data record identifying the determined location of the non-registered used device; and storing or outputting the second location data record. The method further comprises receiving a second type of communication signal data object comprising an identifier of a non-registered user device, an identifier of the registered user device, and a measure of signal strength of a communication signal emitted by the non-registered user device and received by the registered user device; determining a location of the non-registered user device based on the known location of the registered user device and the measure of the signal strength of the communication signal emitted by the non-registered user device and received by the registered user device; generating a second location data record identifying the determined location of the non-registered used device; and storing or outputting the second location data record. The known location of the registered user device is based on GPS location information for the registered user device. The known location of the registered user device is based on the proximity of the registered user device to the network device. The storing or outputting the first location data record comprises at least one of: generating a report identifying respective locations of a plurality of registered or non-registered user devices; generating a map of a facility or area identifying the respective locations of the plurality of registered or non-registered user devices; generating an alert; and generating a report identifying one or more registered or non-registered user devices with which a target registered or non-registered user device has come into a proximity. The communication signal is a passive communication signal comprising at least one of: a BLUETOOTH signal; a BLUETOOTH Low Energy (BLE) signal; a ZigBee signal; a Z-Wave signal; a WiFi signal; a Radio Frequency signal; a cellular signal; and a Near Field Communication (NFC) signal. The network device is one of a plurality of interconnected network devices distributed within or around a facility.

### BRIEF DESCRIPTION OF THE DRAWINGS

FIG. 1 illustrates an overview of an example implementation as described herein.

FIG. 2 illustrates an example environment as described herein.

FIG. 3 illustrates an example flowchart of a process for logging a rich set of location information for registered and non-registered user devices using interconnected network devices in accordance with aspects of the present disclosure.

FIG. 4 shows an example report for close interactions between residents, staff, and visitors in the environment in accordance with aspects of the present disclosure.

FIG. 5 shows an example report or graphic of location history for residents, staff, and visitors in the environment in accordance with aspects of the present disclosure.

FIG. 6 shows an example report 600 for quarantine/ward management in the environment in accordance with aspects of the present disclosure.

FIG. 7 shows an example report 700 for real-time positioning and contract tracing in the environment in accordance with aspects of the present disclosure.

FIG. 8 shows an example report for contract tracing and location history in the environment in accordance with aspects of the present disclosure.

FIG. 9 shows an example report for contact tracing and interactions in the environment in accordance with aspects of the present disclosure.

FIG. 10 shows another example report for contact tracing and interactions in the environment in accordance with aspects of the present disclosure.

FIG. 11 illustrates example components of a device that may be used within environment of FIG. 2.

### DETAILED DESCRIPTION

Aspects of the present disclosure, described herein, may include a system and/or method that logs a rich set of data including the precise locations of user devices at various times. More specifically, the systems and/or methods, described herein, may log the precise locations of user devices that are registered to a location tracing system having multiple distributed network devices at known locations (e.g., fixed locations in or around a facility, such as a nursing home facility or other type of facility). For example, the location of a registered user device may be determined based on a communication signal strength of passive communication signals received by one or more known-location network devices and emitted by the registered user device. More specifically, the distance between the registered user device and the one or more network devices may be determined based on a communication signal strength received by one or more known-location network devices and emitted by the registered user device.

In some embodiments, the communication signals emitted by the registered user device and received by the one or more network devices may include one or more of any variety of network communication signals, such as BLUETOOTH, BLUETOOTH Low Energy (BLE), Near Field Communication (NFC), Z-Wave, ZigBee, Radio Frequency (RF) signals, WiFi, cellular (e.g., Global System for Mobile (GSM) communication, Code Division Multiplex Access (CDMA), Time Division Multiplexing (TDM), Wireless in Local Loop (WILL), or General Packet Radio Services (GPRS) over 3G, 4G, 5G networks), or the like with or without Global Positioning System (GPS) location information. Thus, as the registered user device moves (e.g., as a user wearing or carrying the user device moves), the signals emitted by the registered user device may be received by different network devices with different levels of strength. By using signal strength information, the precise distance between the user device and the network device may be determined (e.g., to within a few feet). Thus, as the location of the network device is known, the precise location of the user device may be determined based on its proximity to (e.g., distance from) the network device.

In one specific illustrative example, the registered user device may be a wearable device (e.g., smart watch, smart pendant, etc.), and the network devices may be interconnected in or around a facility (e.g., a nursing home). In this example, a user may be wearing the user device and as the user moves throughout the facility, the user's precise location may be tracked as the different network devices receive the passive communication signals emitted by the user device. Data records identifying the users precise location at configurable regular time intervals (e.g., each second, every few seconds, every few minutes, etc.) may be generated and stored, and later used to generate any variety of reports, graphical displays of the user's location, on a map or layout, etc.

Each network device that receives a communication signal from a registered user device may provide a data object to a location tracing server in which each data object includes an identifier of the registered user device (which may also correspond to an identifier of a user), an identifier of the network device (e.g., indicating the location of the network device), a signal strength of the received communication signal, and/or a timestamp. The network device may continue to provide data objects at any interval (e.g., every second, every few seconds, every minute, etc.). In this way, the location tracing server may store and log a rich set of location information identifying the precise location of user devices (and hence, the users) at specific times. This rich set of location information may be used to generate any variety of reports or graphical representations of the user's location at different times, the user's proximity to other individuals (e.g., individuals that may have been exposed to an infection), generate alerts when a user exists a defined boundary (e.g., a quarantine boundary, a safe-zone boundary), etc.

In addition to logging the location of registered user devices, the systems and/or methods may also log the locations of non-registered user devices based on their proximity to registered user devices. Specifically, a registered user device may receive passive communication signals from a non-registered user device, and the registered user device may determine a distance between the registered user device and the non-registered user device and report this distance to the location tracing server via the one or more network devices. In this way, the location tracing server may track and log the precise locations of non-registered user devices without requiring any action or software installations on the non-registered user devices. Additionally, or alternatively, the network devices may receive passive communication signals from the non-registered user devices in a similar manner as is done with the registered user devices. When a network device receives a passive communication signal from a non-registered user device, the network device may provide a data object identifying a hardware identifier of the non-registered user device (e.g., a Media Access Control (MAC) address). In this way, user information may be anonymous while still allowing the location tracing server to track the precise locations and presence of user devices, without tracking or storing any user information. As such, user device presence location may be tracked while preserving user anonymity.

Certain embodiments of the disclosure will hereafter be described with reference to the accompanying drawings, wherein like reference numerals denote like elements. It should be understood, however, that the accompanying drawings illustrate only the various implementations described herein and are not meant to limit the scope of various technologies described herein. The drawings show and describe various embodiments of the current disclosure.

Embodiments of the disclosure may include a system, a method, and/or a computer program product at any possible technical detail level of integration. The computer program product may include a computer readable storage medium (or media) having computer readable program instructions thereon for causing a processor to carry out aspects of the present disclosure.

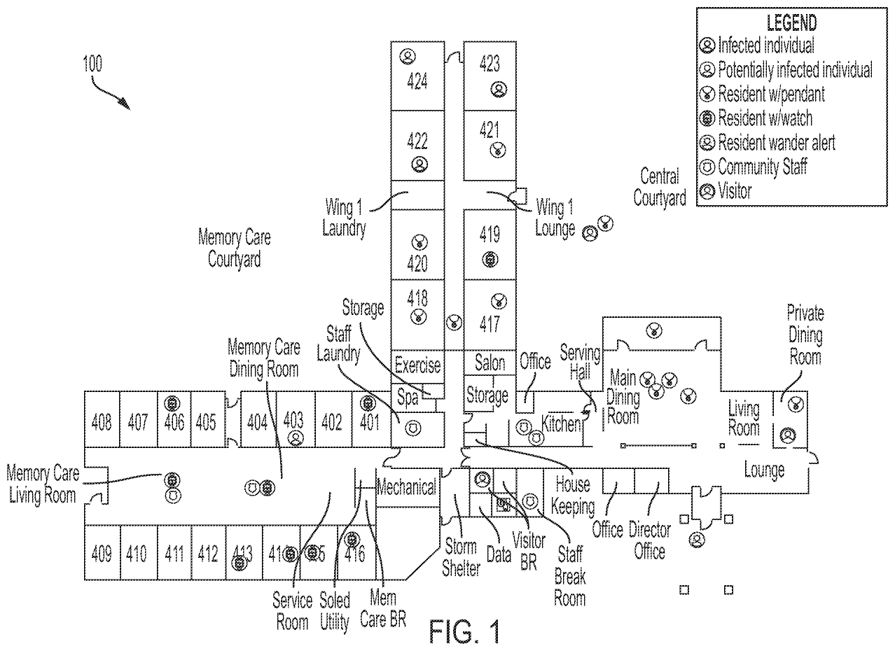

FIG. 1 illustrates an overview of an example implementation in accordance with aspects of the present disclosure. As shown in FIG. 1, a user interface 100 may present location information for users based on the location data received from network devices distributed across a facility (e.g., nursing home facility, hospital facility, or other type of facility). In some embodiments, the user interface 100 may present information identifying individuals that have come in contact with other individuals based on the location data logged and stored by a contact tracing system. In some embodiments, the user interface 100 may present location information in the form of a building map or floor plan with icons representing the location of individuals based on the location of their corresponding user devices (e.g., a real-time location and/or historical location).

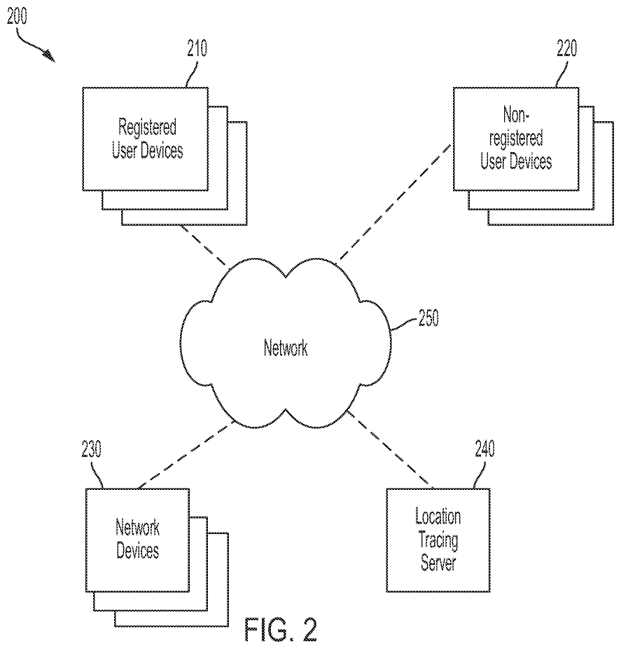

FIG. 2 illustrates an example environment in accordance with aspects of the present disclosure. As shown in FIG. 2, environment 200 includes registered user devices 210, non-registered user device 220, network devices 230, contact tracing server 240, and a network 250. In some embodiments, the registered user device 210, the non-registered user device 220, and/or the network device 230 may be Internet-of-Things (IoT) devices and/or perform one or more functions of IoT devices.

The registered user device 210 may include a computing device capable of communicating via a network, such as the network 240. In example embodiments, the user device 210 corresponds to a mobile communication device (e.g., a smart phone or a personal digital assistant (PDA)), a portable computer device (e.g., a laptop or a tablet computer), a wearable computing device, and/or another type of computing device. In some embodiments, the user device 210 may emit passive communications signals (e.g., BLUETOOTH, BLE, NFC, ZigBee, Z-Wave, WiFi, RF, cellular (e.g., Global System for Mobile (GSM) communication, Code Division Multiplex Access (CDMA), Time Division Multiplexing (TDM), Wireless in Local Loop (WILL), or General Packet Radio Services (GPRS) over 3G, 4G, 5G networks), or the like with or without Global Positioning System (GPS) location information) which may be received by one or more network devices 230. In some examples, the registered user device 210 can change communication protocols based on the location of the user. For example, when the registered user device 210 is location within the environment 200, the registered user device 210 can communicate using a first communication protocol, such as, but is not limited to, BLUETOOTH, ZigBee and its variants, WiFi and its variants, or other short or medium range communication protocols. When the register user device 210 leaves the environment 200, the registered user device 210 can seamlessly and without any user action, change communication protocols to a second communication protocol, such as, but is not limited to, cellular and its variant, or other medium or long range communication protocols with or without GPS location information.

In some embodiments, the registered user device 210 may include a software component that links information regarding the registered user device 210 to the contact tracing server 240 (with the expressed permission of the user). For example, the linked information may include user information, user medical information, user proximity restrictions, user preferences, etc. In some embodiments, the linked information may be provided on an opt-in basis in which the user provides expressed permission to share the linked information. Further, the user may select to opt-out at any time to have their information unlinked from the contact tracing server 240.

The non-registered user device 220 may include any type of user device similar to a registered user device 210. Unlike the registered user device 210, the non-registered user device 220 may not be registered to or have information linked with the contact tracing server 240.

The network device 230 may include one or more network communication devices that may form an interconnected “mesh” network. In some embodiments, multiple network devices 230 may be distributed across a facility (e.g., a nursing home facility or other type of facility). In some embodiments, the network devices 230 may be fixed or have known locations. The network device 230 may be an IoT device, such as a beacon, a smart device, a sensor, or the like. In one embodiment, the network device 230 may be a smart soap/sanitizing agent dispenser equipped with sensors for detecting soap/sanitizing agent levels and/or soap/sanitizing agent usage by a user (e.g., in which the user is identified by a connection between the user's registered user device 210). The soap/sanitizing agent usage may be used to track and/or implement infection control measures by users. The soap/sanitizing agent usage by user may also be stored along with location information by the location tracing server 240.

In some embodiments, a network device 230 may receive passive communication signals from registered user devices 210 and/or non-registered user devices 220. Based on receiving a passive communication signal, the network device 230 may provide, to the contact tracing server 240, a location data object to the contact tracing server 240 that identifies an identifier of the registered user device 210 or the non-registered user device 220 from which the communication signal was received (e.g., a MAC address), an identifier of the network device 230, and a timestamp.

The contact tracing server 240 may include one or more computing systems that receives location data objects from the network devices 230. Based on a receiving a location data object, the contact tracing server 240 may generate and log a location data record that maps the MAC address to an identifier of a registered user device 210 and a corresponding user (e.g., if the location data object is generated from a communication signal received from a registered user device 210). Further, the data record may identify the location of the network device 230 associated with the location data object, and the communication signal strength indicating a distance of the registered user device 210 or the non-registered user device 220 to the network device 230. In this way, a location data record may identify the user device 210 (and a user, if registered). In some embodiments, the contact tracing server 240 may receive a location data object from a registered user device 210 indicating the location of the registered user device 210 (e.g., from a GPS device of the registered user device 210) and the distance between the registered user device 210 and a non-registered user device 220 (e.g., based on a the signal strength of signals emitted by the non-registered user device 220 and received by the registered user device 210).

The network 250 may include network nodes and one or more wired and/or wireless networks. For example, the network 250 may include a cellular network (e.g., a second generation (2G) network, a third generation (3G) network, a fourth generation (4G) network, a fifth generation (2G) network, a long-term evolution (LTE) network, a global system for mobile (GSM) network, a code division multiple access (CDMA) network, an evolution-data optimized (EVDO) network, or the like), a public land mobile network (PLMN), and/or another network. Additionally, or alternatively, the network 250 may include a local area network (LAN), a wide area network (WAN), a metropolitan network (MAN), the Public Switched Telephone Network (PSTN), an ad hoc network, a managed Internet Protocol (IP) network, a virtual private network (VPN), an intranet, the Internet, a fiber optic-based network, and/or a combination of these or other types of networks. In embodiments, the network 250 may include copper transmission cables, optical transmission fibers, wireless transmission, routers, firewalls, switches, gateway computers and/or edge servers.

The quantity of devices and/or networks in the environment 200 is not limited to what is shown in FIG. 2. In practice, the environment 200 may include additional devices and/or networks; fewer devices and/or networks; different devices and/or networks; or differently arranged devices and/or networks than illustrated in FIG. 2. Also, in some implementations, one or more of the devices of the environment 200 may perform one or more functions described as being performed by another one or more of the devices of the environment 200. Devices of the environment 200 may interconnect via wired connections, wireless connections, or a combination of wired and wireless connections.

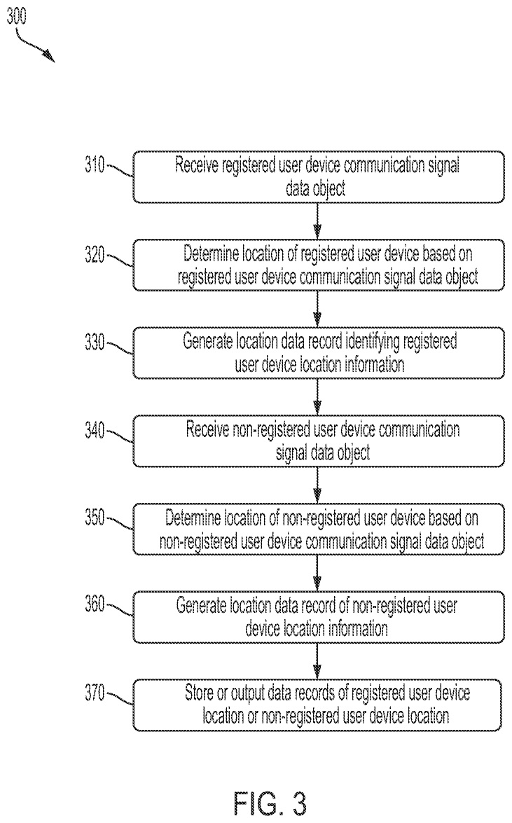

FIG. 3 illustrates an example flowchart of a process for logging a rich set of location information for registered and non-registered user devices using interconnected network devices in accordance with aspects of the present disclosure. The blocks of FIG. 3 may be implemented in the environment of FIG. 2, for example, and are described using reference numbers of elements depicted in FIG. 2. As noted herein, the flowchart illustrates the architecture, functionality, and operation of possible implementations of systems, methods, and computer program products according to various embodiments of the present disclosure.

As shown in FIG. 3, The process 300 may include receiving registered user device communication signal data object (block 310). For example, the contact tracing server 240 may receive a registered user device communication signal data object associated with a registered user device 210 from a network device 230. In some embodiments, the registered user device communication signal data object may identify the network device 230, the registered user device 210, and a measure of the strength of the communication signal received by the network device 230 and emitted by the registered user device 210.

The process 300 also may include determining a location of the registered user device based on the registered user device communication signal data object (block 320). For example, the contact tracing server 240 may determine the location of the registered user device 210 based on the measure of the strength of the communication signal received by the network device 230 and emitted by the registered user device 210 (e.g., as identified in the registered user device communication signal data object). In some embodiments, the contact tracing server 240 may determine a radius of the location of the registered user device 210 based on the received signal strength and the known location of the network device 230 (e.g., based on the identifier of the network device 230 included in the registered user device communication signal data object). For example, lower signal strengths may indicate that the distance between the registered user device 210 and the contact tracing server 240 are higher. In some embodiments, the contact tracing server 240 may combine signal strength data from multiple different network devices 230 to determine a more precise location of the registered user device 210.

The process 300 further may include generating a location data record identifying the registered user device location information (block 330). For example, the contact tracing server 240 may generating a location data record identifying the location information for the registered user device 210. In some embodiments, the location data record may identify the registered user device 210, a user registered to the registered user device 210, and/or a radius of location of the registered user device 210 (e.g., as determined at step 330), etc.

The process 300 also may include (block 340). For example, the contact tracing server 240 may receiving a non-registered user device communication signal data object (block 310). For example, the contact tracing server 240 may receive a non-registered user device communication signal data object associated with a non-registered user device 220 from a network device 230. In some embodiments, the network device 230 may directly receive passive communication signals from the non-registered user device 220 and generate and provide the non-registered user device communication signal data object to the contact tracing server 240. Additionally, or alternatively, the contact tracing server 240 may receive a non-registered user device communication signal data object from a registered user device 210 (e.g., via the network device 230). In some embodiments, the non-registered user device communication signal data object may identify a signal strength emitted by the non-registered user device 220 and received by the registered user device 210.

The process 300 further may include determining the location of the non-registered user device based on the non-registered user device communication signal data object (block 330). For example, the contact tracing server 240 may determine the location of the non-registered user device 220. More specifically, since the location of the registered user device 210 may be determined (e.g., as described in process blocks 310-330), the location of the non-registered user device 220 may be determined based on the proximity of the non-registered user device 220 to the registered user device 210 (e.g., based on a measure of the strength of the passive signal emitted by the non-registered user device 220 and received by the registered user device 210). Additionally, or alternatively, the location of the non-registered user device 220 may be determined based on the proximity of the non-registered user device 220 to the network device 230 (e.g., based on a measure of the strength of the passive signal emitted by the non-registered user device 220 and received by the network device 230).

The process 300 also may include generating a location data record identifying the non-registered user device location information (block 360). For example, the contact tracing server 240 may generating a location data record identifying the location information for the non-registered user device 220. In some embodiments, the location data record may identify the non-registered user device 220 (e.g., a MAC address of the non-registered user device 220), and/or a radius of location of the registered user device 210 (e.g., as determined at step 340). In some examples, the location data record can list all or less than all of the places an individual went either with the non-registered user device 220 and/or the registered user device 210. Also, the location data record can list how much time individuals spent in each location and/or track the path of every individual took with the non-registered user device 220 and/or the registered user device 210 within the environment 200.

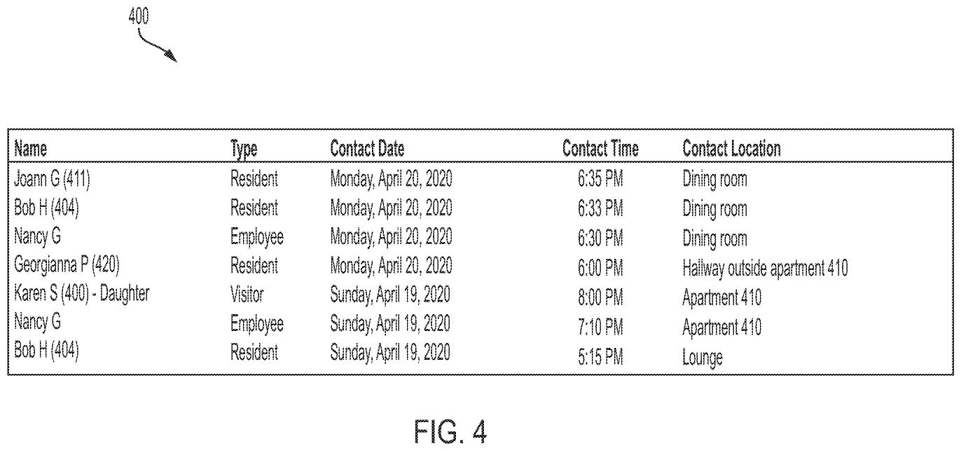

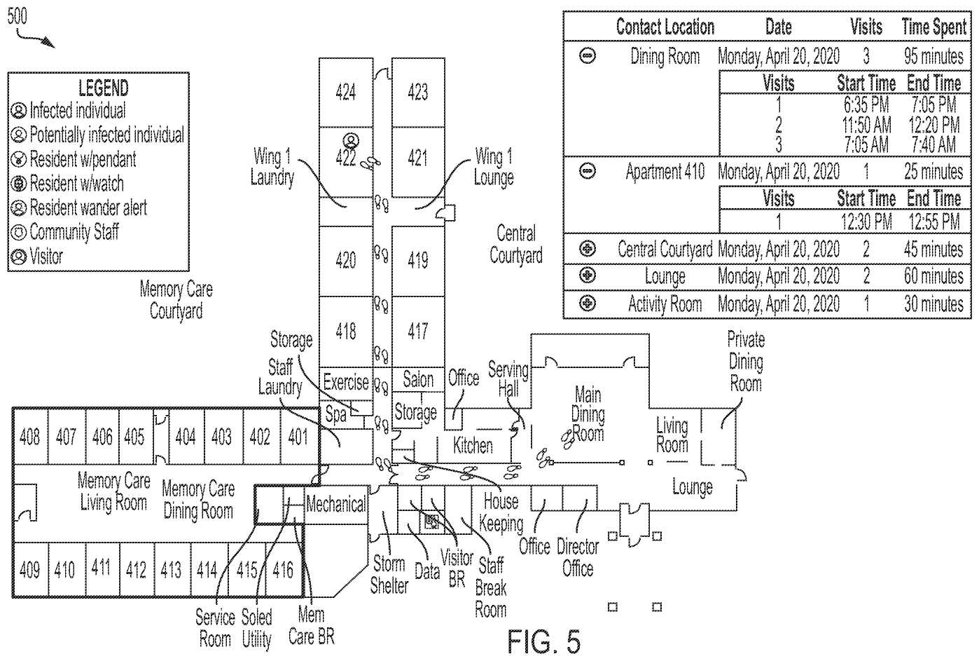

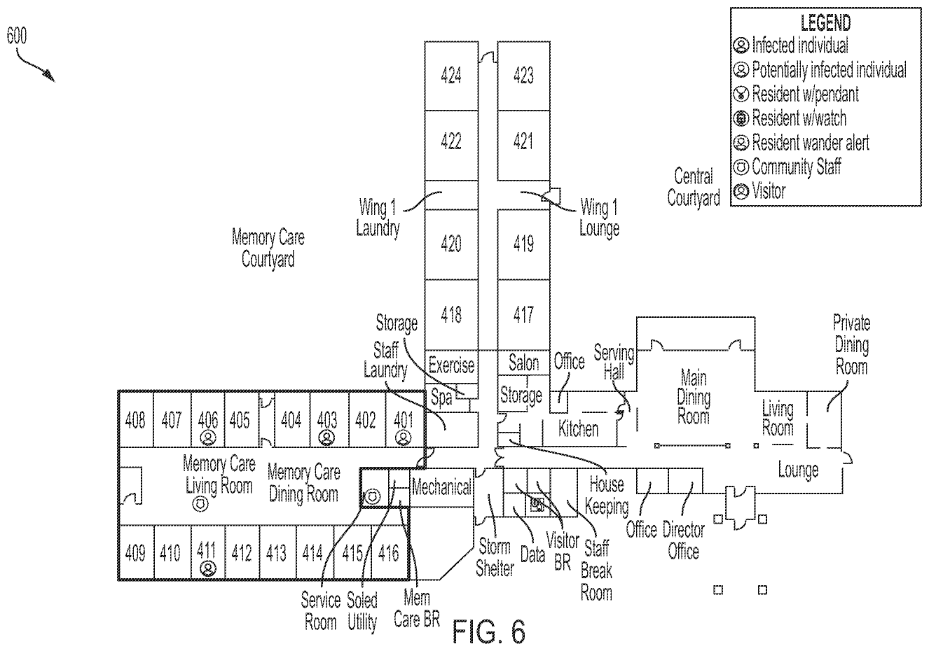

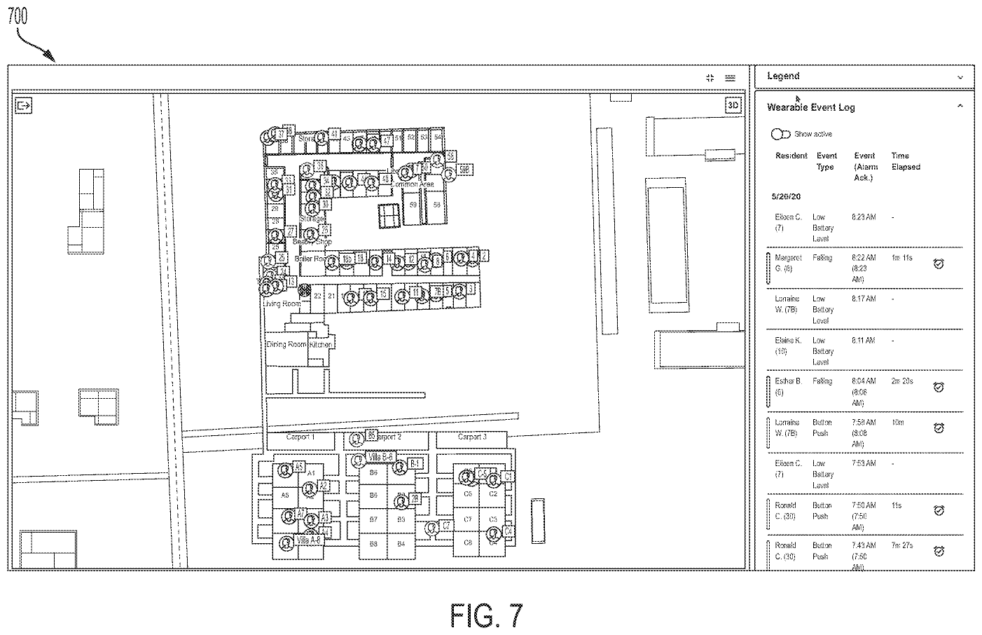

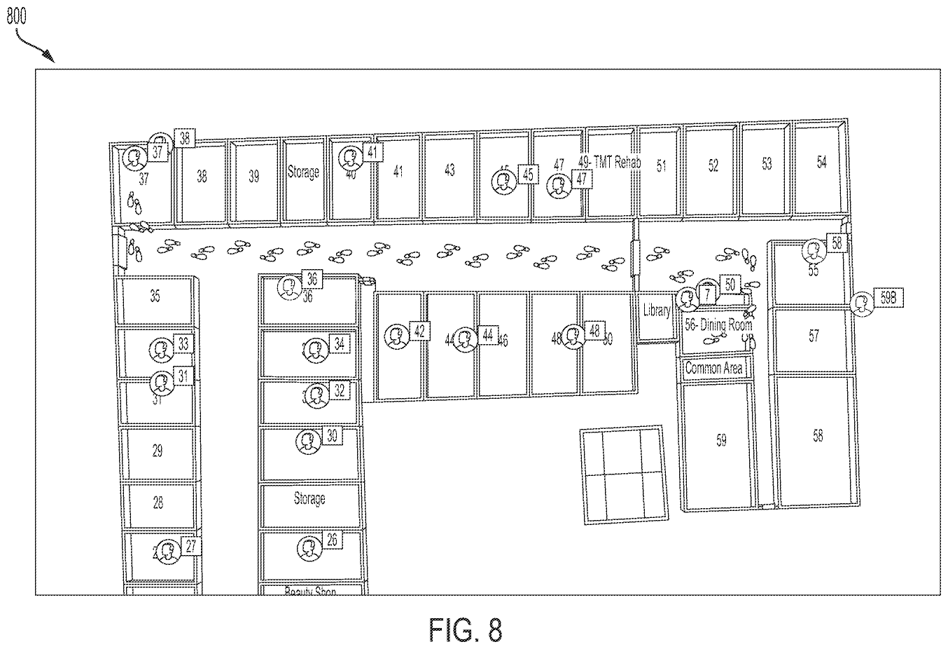

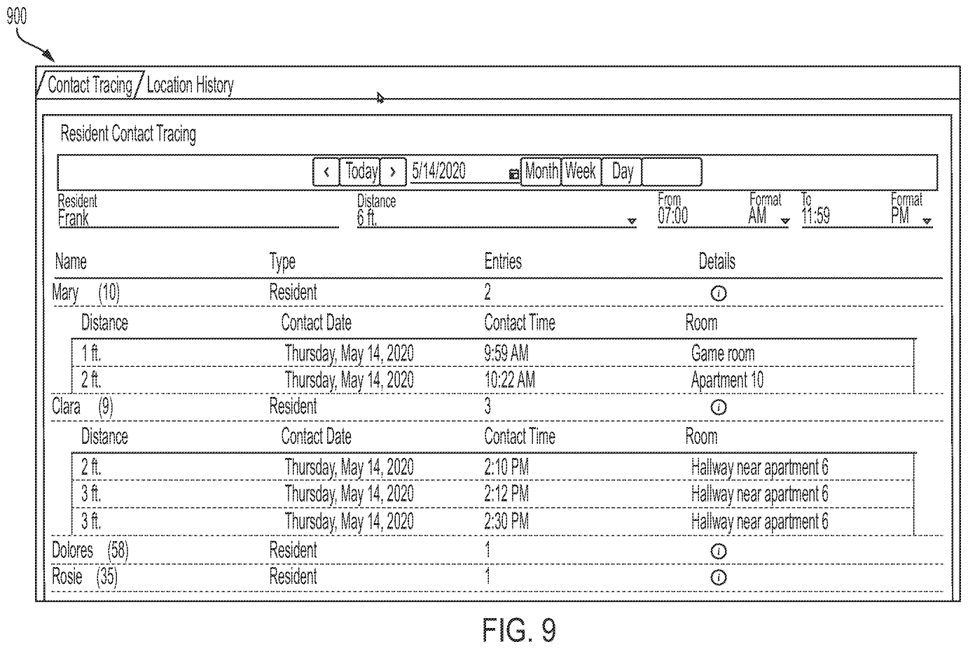

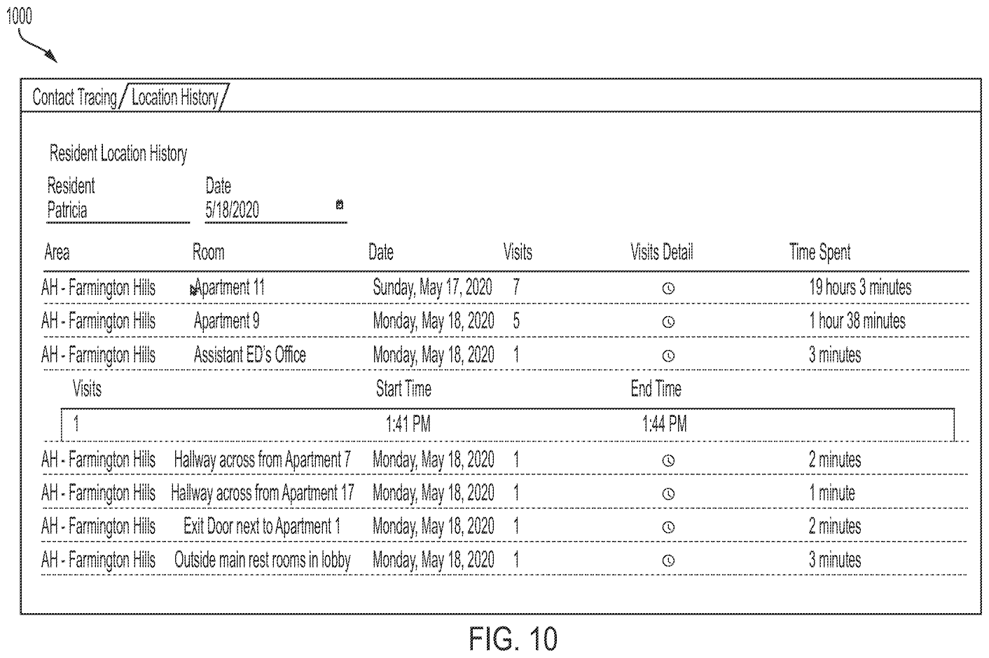

The process 300 further may include storing or outputting data records of registered user device location or non-registered user device location (block 370). For example, the contact tracing server 240 may store or output data records of registered user device location or non-registered user device location. In some embodiments, the contact tracing server 240 may output or present the data records in the form of a chart, data structure, and/or map (e.g., floor layout, such as that shown in the interface 100 of FIG. 1). In some embodiments, the data records may be used to generate any variety of reports, such as reports indicating the location of a user at a given time, reports indicating other users with whom a target has been in contact, alerts for wander management indicating that a user has exited or is close to exiting a particular boundary (such as an alert when individuals break quarantine or a quarantined area), etc. FIG. 4 shows an example report 400 for close interactions between residents, staff, and visitors in the environment 200. The report 400 can assist with knowing with whom individuals have come into contact, monitoring potential spread of a disease from person-to-person, and/or identifying possible areas to quarantine. FIG. 5 shows an example report or graphic 500 of location history for residents, staff, and visitors in the environment 200. The report 500 can list all the places an individual went, show how much time individuals spent in each location, and/or track the path every individual took within the environment 200. FIG. 6 shows an example report 600 for quarantine/ward management in the environment 200. The report 600 can be used to reduce the spread of illness and monitor for developments of the illness/disease by allowing users to receive alerts when individuals break the quarantine area, monitor wander risk and be alerted to potential developments, and/or customize alerts for residents, staff, and visitors. FIG. 7 shows an example report 700 for real-time positioning and contract tracing in the environment 200. FIG. 8 shows an example report 800 for contract tracing and location history in the environment 200. FIG. 9 shows an example report 900 for contact tracing and interactions in the environment 200. FIG. 10 shows another example report 1000 for contact tracing and interactions in the environment 200.

In some embodiments, the process 300 may be repeated (e.g., every second, few seconds, every minute, etc.). In this way, the contact tracing server 240 may generate and store a rich set of data identifying the precise locations of both registered user devices 210 and non-registered user devices 220. This rich set of data may be used to generate any variety of reports, alerts, etc., as described herein.

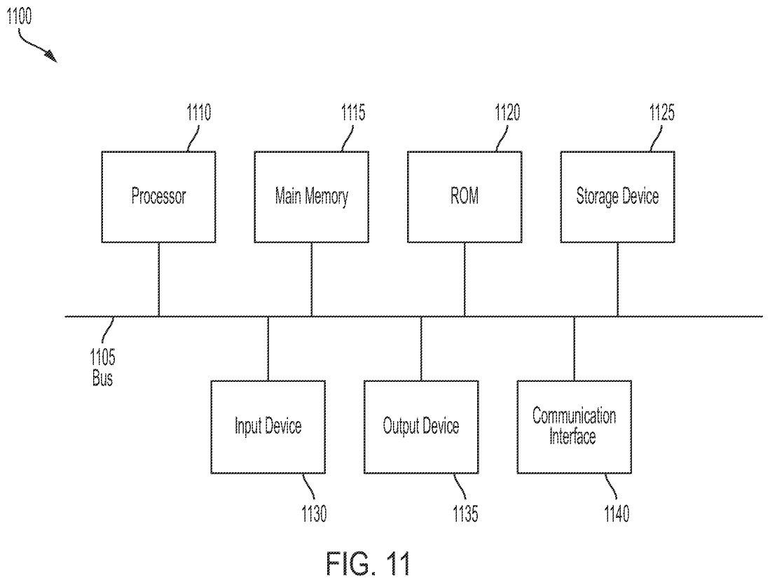

FIG. 11 illustrates example components of a device 1100 that may be used within environment 200 of FIG. 2. Device 1100 may correspond to registered user device 210, the non-registered user device 220, the network device 230, and/or the contact tracing server 240. Each of registered user device 210, the non-registered user device 220, the network device 230, and/or the contact tracing server 240 may include one or more devices 400 and/or one or more components of device 400.

As shown in FIG. 11, device 1100 may include a bus 1105, a processor 1110, a main memory 1115, a read only memory (ROM) 1120, a storage device 1125, an input device 1130, an output device 1135, and a communication interface 1140.

Bus 1105 may include a path that permits communication among the components of device 1100. Processor 1110 may include a processor, a microprocessor, an application specific integrated circuit (ASIC), a field programmable gate array (FPGA), or another type of processor that interprets and executes instructions. Main memory 1115 may include a random access memory (RAM) or another type of dynamic storage device that stores information or instructions for execution by processor 1110. ROM 1120 may include a ROM device or another type of static storage device that stores static information or instructions for use by processor 1110. Storage device 1125 may include a magnetic storage medium, such as a hard disk drive, or a removable memory, such as a flash memory.

Input device 1130 may include a component that permits an operator to input information to device 1100, such as a control button, a keyboard, a keypad, or another type of input device. Output device 1135 may include a component that outputs information to the operator, such as a light emitting diode (LED), a display, or another type of output device. Communication interface 1140 may include any transceiver-like component that enables device 1100 to communicate with other devices or networks. In some implementations, communication interface 1140 may include a wireless interface, a wired interface, or a combination of a wireless interface and a wired interface. In embodiments, communication interface 1140 may receiver computer readable program instructions from a network and may forward the computer readable program instructions for storage in a computer readable storage medium (e.g., storage device 1125).

Device 1100 may perform certain operations, as described in detail below. Device 1100 may perform these operations in response to processor 1110 executing software instructions contained in a computer-readable medium, such as main memory 1115. A computer-readable medium may be defined as a non-transitory memory device and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. A memory device may include memory space within a single physical storage device or memory space spread across multiple physical storage devices.

The software instructions may be read into main memory 1115 from another computer-readable medium, such as storage device 1125, or from another device via communication interface 1140. The software instructions contained in main memory 1115 may direct processor 1110 to perform processes that will be described in greater detail herein. Alternatively, hardwired circuitry may be used in place of or in combination with software instructions to implement processes described herein. Thus, implementations described herein are not limited to any specific combination of hardware circuitry and software.

In some implementations, device 1100 may include additional components, fewer components, different components, or differently arranged components than are shown in FIG. 11.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the disclosure may include a system, a method, and/or a computer program product at any possible technical detail level of integration. The computer program product may include a computer readable storage medium (or media) having computer readable program instructions thereon for causing a processor to carry out or execute aspects and/or processes of the present disclosure.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on a user's computer, partly on the user's computer, as a stand-alone software package, partly on the user's computer and partly on a remote computer or entirely on the remote computer or server.

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In embodiments, a service provider could offer to perform the processes described herein. In this case, the service provider can create, maintain, deploy, support, etc., the computer infrastructure that performs the process steps of the disclosure for one or more customers. These customers may be, for example, any business that uses technology. In return, the service provider can receive payment from the customer(s) under a subscription and/or fee agreement and/or the service provider can receive payment from the sale of advertising content to one or more third parties.

The foregoing description provides illustration and description, but is not intended to be exhaustive or to limit the possible implementations to the precise form disclosed. Modifications and variations are possible in light of the above disclosure or may be acquired from practice of the implementations.

It will be apparent that different examples of the description provided above may be implemented in many different forms of software, firmware, and hardware in the implementations illustrated in the figures. The actual software code or specialized control hardware used to implement these examples is not limiting of the implementations. Thus, the operation and behavior of these examples were described without reference to the specific software code—it being understood that software and control hardware can be designed to implement these examples based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While the present disclosure has been disclosed with respect to a limited number of embodiments, those skilled in the art, having the benefit of this disclosure, will appreciate numerous modifications and variations there from. It is intended that the appended claims cover such modifications and variations as fall within the true spirit and scope of the disclosure.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

What is claimed is:

1. A computer-implemented method comprising:
receiving, by a computing device, a first type of communication signal data object comprising an identifier of a registered user device, an identifier of a network device, and a measure of signal strength of a communication signal emitted by the registered user device and received by the network device;
determining, by the computing device, a location of the registered used device based on a known location of the network device and the measure of the signal strength;
generating, by the computing device, a first location data record identifying the location that is determined of the registered used device and a user registered to the registered user device; and
storing or outputting, by the computing device, the first location data record.

2. The method of claim 1, further comprising:
receiving a second type of communication signal data object comprising an identifier of a non-registered user device, an identifier of the network device, and a measure of signal strength of a communication signal emitted by the non-registered user device and received by the network device;
determining a location of the non-registered user device based on the known location of the network device and the measure of the signal strength of the communication signal emitted by the non-registered user device and received by the network device;
generating a second location data record identifying the determined location of the non-registered used device; and
storing or outputting the second location data record.

3. The method of claim 1, further comprising:
receiving a second type of communication signal data object comprising an identifier of a non-registered user device, an identifier of the registered user device, and a measure of signal strength of a communication signal emitted by the non-registered user device and received by the registered user device;
determining a location of the non-registered user device based on the known location of the registered user device and the measure of the signal strength of the communication signal emitted by the non-registered user device and received by the registered user device;
generating a second location data record identifying the determined location of the non-registered used device; and
storing or outputting the second location data record.

4. The method of claim 3, wherein the known location of the registered user device is based on GPS location information for the registered user device.

5. The method of claim 3, wherein the known location of the registered user device is based on a proximity of the registered user device to the network device.

6. The method of claim 1, wherein the storing or outputting the first location data record comprises at least one of:
generating a report identifying respective locations of a plurality of registered or non-registered user devices;
generating a map of a facility or area identifying the respective locations of the plurality of registered or non-registered user devices;
generating an alert; and
generating a report identifying one or more registered or non-registered user devices with which a target registered or non-registered user device has come into a proximity.

7. The method of claim 1, wherein the communication signal is a passive communication signal comprising at least one of:
a BLUETOOTH signal;
a BLUETOOTH Low Energy (BLE) signal.
a ZigBee signal;
a Z-Wave signal;
a WiFi signal;
a Radio Frequency signal;
a cellular signal; and
a Near Field Communication (NFC) signal.

8. The method of claim 1, wherein the network device is one of a plurality of interconnected network devices distributed within or around a facility.

9. A computer program product comprising a computer readable storage medium having program instructions embodied therewith, the program instructions executable by a computing device to cause the computing device to perform a method comprising:
receiving, by the computing device, a first type of communication signal data object comprising an identifier of a registered user device, an identifier of a network device, and a measure of signal strength of a communication signal emitted by the registered user device and received by the network device;
determining, by the computing device, a location of the registered used device based on a known location of the network device and the measure of the signal strength;
generating, by the computing device, a first location data record identifying the location that is determined of the registered used device and a user registered to the registered user device; and
storing or outputting, by the computing device, the first location data record.

10. The computer program product of claim 9, wherein the computing device further performs the method comprising:
receiving a second type of communication signal data object comprising an identifier of a non-registered user device, an identifier of the network device, and a measure of signal strength of a communication signal emitted by the non-registered user device and received by the network device;
determining a location of the non-registered user device based on the known location of the network device and the measure of the signal strength of the communication signal emitted by the non-registered user device and received by the network device;
generating a second location data record identifying the determined location of the non-registered used device; and
storing or outputting the second location data record.

11. The computer program product of claim 9, wherein the computing device further performs the method comprising:
receiving a second type of communication signal data object comprising an identifier of a non-registered user device, an identifier of the registered user device, and a measure of signal strength of a communication signal emitted by the non-registered user device and received by the registered user device;
determining a location of the non-registered user device based on the known location of the registered user device and the measure of the signal strength of the communication signal emitted by the non-registered user device and received by the registered user device;
generating a second location data record identifying the determined location of the non-registered used device; and
storing or outputting the second location data record.

12. The computer program product of claim 11, wherein the known location of the registered user device is based on GPS location information for the registered user device.

13. The computer program product of claim 11, wherein the known location of the registered user device is based on a proximity of the registered user device to the network device.

14. The computer program product of claim 9, wherein the storing or outputting the first location data record comprises at least one of:
generating a report identifying respective locations of a plurality of registered or non-registered user devices;
generating a map of a facility or area identifying the respective locations of the plurality of registered or non-registered user devices;
generating an alert; and
generating a report identifying one or more registered or non-registered user devices with which a target registered or non-registered user device has come into a proximity.

15. The computer program product of claim 9, wherein the communication signal is a passive communication signal comprising at least one of:
a BLUETOOTH signal;
a BLUETOOTH Low Energy (BLE) signal;
a ZigBee signal;
a Z-Wave signal;
a WiFi signal;
a Radio Frequency signal;
a cellular signal; and
a Near Field Communication (NFC) signal.

16. The computer program product of claim 9, wherein the network device is one of a plurality of interconnected network devices distributed within or around a facility.

17. A system comprising:
a processor, a computer readable memory, a non-transitory computer readable storage medium associated with a computing device, and program instructions executable by the computing device to cause the computing device to perform operations of a method comprising:
receiving, by a computing device, a first type of communication signal data object comprising an identifier of a registered user device, an identifier of a network device, and a measure of signal strength of a communication signal emitted by the registered user device and received by the network device;
determining, by the computing device, a location of the registered used device based on a known location of the network device and the measure of the signal strength;
generating, by the computing device, a first location data record identifying the location that is determined of the registered used device and a user registered to the registered user device; and
storing or outputting, by the computing device, the first location data record.

18. The system of claim 17, wherein the operations further comprise:
receiving a second type of communication signal data object comprising an identifier of a non-registered user device, an identifier of the network device, and a measure of signal strength of a communication signal emitted by the non-registered user device and received by the network device;
determining a location of the non-registered user device based on the known location of the network device and the measure of the signal strength of the communication signal emitted by the non-registered user device and received by the network device;
generating a second location data record identifying the determined location of the non-registered used device; and
storing or outputting the second location data record.

19. The system of claim 17, wherein the operations further comprise:
receiving a second type of communication signal data object comprising an identifier of a non-registered user device, an identifier of the registered user device, and a measure of signal strength of a communication signal emitted by the non-registered user device and received by the registered user device;
determining a location of the non-registered user device based on the known location of the registered user device and the measure of the signal strength of the communication signal emitted by the non-registered user device and received by the registered user device;
generating a second location data record identifying the determined location of the non-registered used device; and
storing or outputting the second location data record.

20. The system of claim 19, wherein the known location of the registered user device is based on GPS location information for the registered user device.
